(19) United States
(12) Patent Application Publication
(10) Pub. No.: US 2024/0188699 A1
(43) Pub. Date: Jun. 13, 2024

(54) INSTRUMENT CONTAINER, CORRESPONDING USE AND METHOD FOR PACKAGING AND/OR TRANSPORTING AN INSTRUMENT IN A HOUSING

(71) Applicant: Hohner Musikinstrumente GmbH, Trossingen (DE)

(72) Inventors: Joachim Bierer, Trossingen (DE); Anne KREMER, Trossingen (DE); Knut HOYER, Villingen-Schwenningen (DE)

(73) Assignee: Hohner Musikinstrumente GmbH, Trossingen (DE)

(21) Appl. No.: 18/530,470

(22) Filed: Dec. 6, 2023

(30) Foreign Application Priority Data
Dec. 7, 2022 (DE) 102022132574.1

(51) Int. Cl. A45C 13/02; A45C 11/24; A61B 50/30; G10G 7/00

(52) U.S. Cl. CPC A45C 13/02; A45C 11/24; A61B 50/30; G10G 7/00

(57) ABSTRACT

An instrument holder (1) which provides for the flexible holding of different instruments (3) within a housing (2). The instrument holder includes at least one spring clip (4-12) which contacts and/or acts upon the instrument (3) inserted in each case.

### TECHNICAL FIELD

The invention relates to an instrument container.

### BACKGROUND

Instrument containers are known in practice and often have a shape-adapted insert, which is exactly or at least approximately adapted to the instrument in question to be accommodated. Instrument containers are used, for example, for musical instruments or other, in particular shock-sensitive instruments, also measuring instruments or surgical instruments, for transport and/or for protected storage.

### SUMMARY

The invention is based on the object of designing an instrument container with improved use and/or production properties.

To achieve the object mentioned, one or more of the features disclosed herein are provided. In particular, it is thus proposed according to the invention for achieving the object mentioned, in the case of an instrument container, to equip the instrument container with a housing and at least one spring clip which is designed to bear against an inserted instrument. Thus, a secure holding of the instrument in the housing can be achieved in a manner which is easy to manufacture, even with instruments differing in size.

A preferred application is if the instrument is a musical instrument, for example a harmonica.

In an advantageous refinement, it can be provided that the at least one spring clip is strip-shaped at least in a contact portion. Flat contact with the instrument can thus be achieved. This improves holding of the instrument in the housing and an introduction of force into the instrument.

In an advantageous refinement, it can be provided that the at least one spring clip is formed on an inner part. Thus, the at least one spring clip is positionable and also mountable independently of the housing.

In an advantageous refinement, it can be provided that the at least one spring clip is arranged removably on the housing. Thus, contact with the instrument can be made before the instrument is placed in the housing. For example, it can thus be achieved that the instrument is moved into the housing with a spring clip or spring clips in contact with the instrument.

For example, it can be provided for this purpose that the at least one spring clip is formed as part of an inner part, in particular the inner part already mentioned. This allows simultaneous removal of a plurality of spring clips. It is also possible to position the at least one spring clip relative to the housing and/or to further spring clips via the inner part.

In an advantageous refinement, it can be provided that the at least one spring clip bounds a receiving space for the instrument. A defined storage position for an instrument can thus be created. For example, this receiving space can be surrounded by a buffer zone which is formed within the housing.

This can be, for example, in at least two, preferably at least three or four directions. This allows multi-sided or even all-round protection.

In an advantageous refinement, it can be provided that the at least one spring clip is supported, in particular fastened, at at least two points, in particular at its ends. Thus, a stable holding of the instrument and good dissipation of holding forces can be achieved. For example, the spring clip can be fastened to the inner part.

In an advantageous refinement, it can be provided that the at least one spring clip is formed circumferentially. A complete enclosure of the instrument along a circumference can thus be achieved.

In an advantageous refinement, it can be provided that the at least one spring clip is formed on a grid. Intersecting structures can thus be formed, for example by intersecting spring clips. This can facilitate all-round protection.

For example, the spring clip can be formed on a cage. A surface of the instrument is thus contactable, in particular fixable, by a plurality of identically extending spring clips.

In an advantageous refinement, it can be provided that the spring clip is a first spring clip and that there is at least one further spring clip which is designed to bear against an inserted instrument. The instrument is thus fixable at a plurality of locations, for example, spaced apart from one another.

In this case, it can be provided, for example, that the further spring clip is connected to the first spring clip preferably nonreleasably, in particular in an integrally bonded manner. Thus, particularly stiff arrangements of spring clips can be formed in a simple way.

Alternatively or additionally, it can be provided here that the first spring clip and the further spring clip are arranged in the housing in such a way that they clamp each other over an inserted instrument. This means that the instrument can be accommodated securely between two spring clips.

In an advantageous refinement, it can be provided that the at least one spring clip is supported on the housing. An introduction of force into the housing can thus be achieved. This can also be used to provide effective protection against external impacts.

In an advantageous refinement, it can be provided that the at least one spring clip spans a distance between the housing and an inserted instrument. A buffer zone, for example a cavity, can thus be formed and maintained between the instrument and the housing.

In an advantageous refinement, it can be provided that the at least one spring clip has a plastics surface. Thus, scratching of the instrument as it is being pushed into the arrangement of at least one spring clip, for example into an inner part, in particular the inner part already described, can be avoided.

In an advantageous refinement, it can be provided that the or an inner part having the at least one spring clip is formed integrally and/or as an injection-molded part. A one-piece design can be advantageous for installation, as fewer installation steps are required. A configuration as an injection-molded part enables manufacturing costs to be reduced. Another advantage is a comparatively large selection of materials. Thus, for example, a surface finish and/or a stiffness can be adapted to the instruments to be accommodated.

In an advantageous refinement, it can be provided that the housing is dimensionally stable. A stable enclosure of the instrument can thus be created.

In an advantageous refinement, it can be provided that the housing has at least two housing shells. The housing can thus be easily opened.

It can be provided in this connection that the housing shells are connected to each other via a hinge. This allows opening without the risk of losing one of the housing shells, especially when opening with one hand.

In an advantageous refinement, it can be provided that at least one housing shell of the housing forms more than a half of a surface of the housing. It can thus be achieved that the at least one housing shell takes over a predominant part of shielding and fixing the inserted instrument. It is also possible for the instrument to be held in the housing shell even when the housing is open.

For example, it can be provided in this connection that the housing shell forms more than two thirds, or more than three quarters, of the surface. The instrument can thus be substantially accommodated in one of the two housing shells, and the risk of the instrument inadvertently falling out when opening the housing can be reduced.

In an advantageous refinement, it can be provided that the at least one spring clip is substantially or completely accommodated by the at least one housing shell. It is thus easily possible for the at least one spring clip to be able to be supported on the housing before the housing is closed. This facilitates one-handed operation.

In this connection, it can be provided, for example, that an inner part, in particular the inner part already mentioned, which comprises or has the spring clip, is substantially or completely accommodated by the at least one housing shell. The inner part with the instrument already accommodated can thus easily be inserted into the housing.

In an advantageous refinement, it can be provided that the at least one spring clip creates a receiving space which holds an inserted instrument on all sides at a distance from the housing.

Alternatively or additionally, it can be provided that the at least one spring clip creates a receiving space which holds an inserted instrument at least over a longitudinal extent of the instrument. Uniform fixing, in particular in the case of elongate instruments, can thus be achieved.

In an advantageous refinement, it can be provided that the container has at least two housing shells which are movable relative to one another. This can facilitate opening.

For example, the housing shells can be pivotable relative to one another. For this purpose, it can be provided, for example, that the housing shells are connected via a hinge. This makes it easy to ensure that the housing shells remain connected even when the housing is open.

A first housing shell can have a holding region for contacting by a hand. This allows manual fixing, for example, for opening and/or closing.

It is particularly favorable if the holding region on the housing is designed for contacting by a palm. This permits a secure hold, in particular for one-handed opening and/or closing of the housing.

Alternatively or additionally, it can be provided that a housing shell has an actuating region for contacting by a finger of the hand. This means that the housing can thus be opened and/or closed with one hand.

For example, this finger may be a thumb of the hand. This permits an ergonomic opening and/or closing process.

As an advantageous application of the invention, the use of an instrument container for accommodating instruments of differing shapes and/or differing sizes is proposed.

An advantageous application of the invention can provide a method for packing an instrument into a housing, the instrument being introduced into a receiving space of an inner part which has at least one spring clip, at least the at least one spring clip being deformed, and the instrument then being introduced with the inner part into the housing, with the at least one spring clip being further deformed. A mechanical load on the instrument by the at least one spring clip as the instrument is being inserted can thus be reduced. Simple packing, in particular immediately during or after manufacturing, can be achieved. It can be provided in this connection that the inner part is fixed as it is introduced for the first time into the housing.

Alternatively, it can be provided that the inner part is introduced beforehand into the housing. The inner part can therefore remain, preferably can be fixed, in the housing when packing and removal takes place again.

### BRIEF DESCRIPTION OF THE DRAWINGS

The invention will now be described in more detail on the basis of exemplary embodiments, but is not restricted to the exemplary embodiments. Further exemplary embodiments emerge from combining the features of individual or multiple claims with one another and/or with individual or multiple features of the exemplary embodiment.

In the figures:

FIG. 1 shows an exploded illustration of an instrument container according to the invention with an instrument,

FIG. 2 shows an inner part of the instrument carrier from FIG. 1 in a view of a wide side with a cut-open housing,

FIG. 3 shows the inner part from FIG. 2 in a view of a narrow side in the cut-open housing,

FIG. 4 shows a view from above of the instrument container from FIG. 1 with the housing open,

FIG. 5 shows the inner part from FIG. 1 in a three-dimensional oblique view,

FIG. 6 shows a further exploded illustration of the instrument container from FIG. 1,

FIG. 7 shows the instrument container from FIG. 1 with an inner part partially inserted,

FIG. 8 shows an alternative inner part,

FIG. 9 shows another alternative inner part,

FIG. 10 shows a further spring clip for use in the instrument container according to FIG. 1,

FIG. 11 shows another alternative inner part,

FIG. 12 shows another alternative inner part with protruding, bristle-like spring clips,

FIG. 13 shows another alternative inner part with a laterally open receiving space,

FIG. 14 shows another alternative inner part with a laterally open receiving space,

FIG. 15 shows a further alternative inner part with a cage closed in the circumferential direction, and

FIG. 16 shows a housing shell with integrally formed spring clips of an instrument container according to the invention.

### DETAILED DESCRIPTION

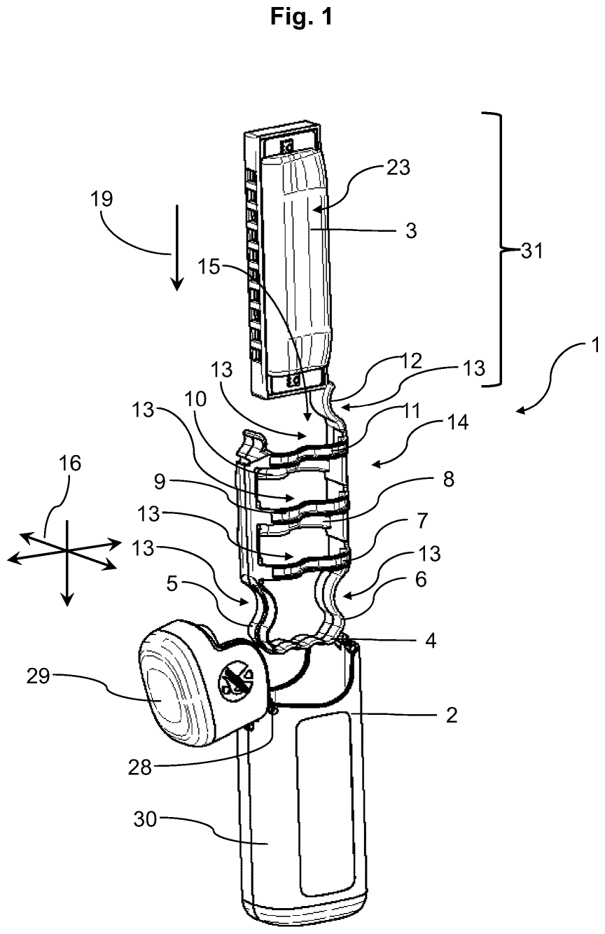

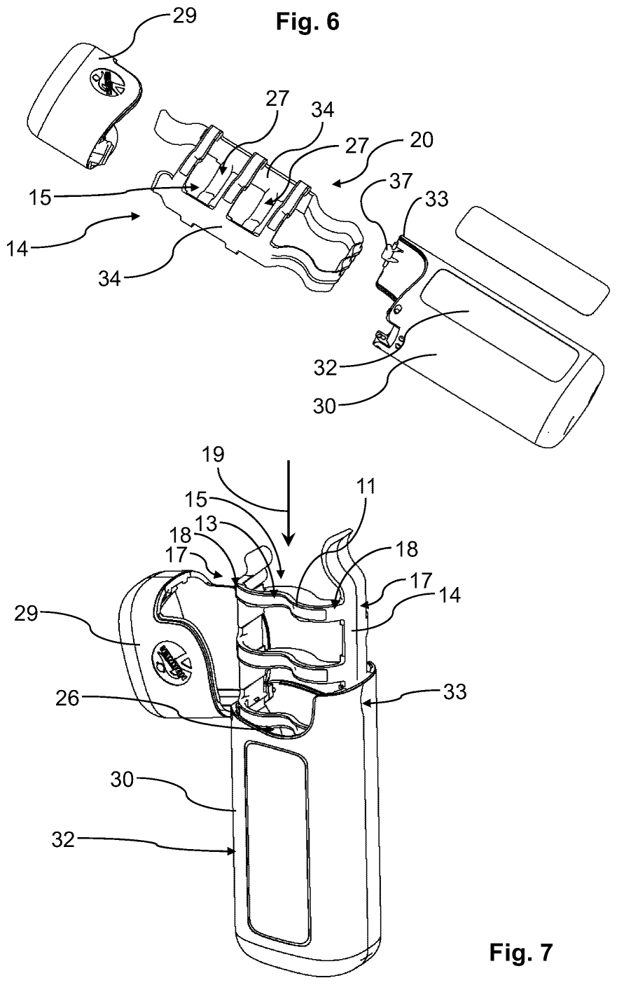

FIGS. 1-7 show a first exemplary embodiment of the invention and will be described together below.

An instrument container, denoted overall by 1, has a housing 2, which is designed for accommodating an instrument 3.

During use or during transport, the housing 2 completely surrounds the instrument 3 and thus protects it from mechanical influences from the outside.

A number of spring clips 4-12 against which the instrument 3 bears during transport or during storage are arranged in the interior of the housing 2.

In this connection, each spring clip 4-12 forms at least one contact portion 13, in which the spring clip 4-12 is placed against the instrument in such a way that the spring clip is slightly deformed by the instrument 3.

The spring clips 4-12 are each strip-shaped, and therefore a strip-shaped contact portion 13 is formed.

The spring clips 4-12 are formed here on an inner part 14.

The inner part 14 is arranged removably on the housing 2 and can be inserted into same or pulled out therefrom.

Holding lugs or latching means (not illustrated specifically) for captively fixing the inner part 14 are formed in the housing 2. The inner part 14 thus remains in the housing 2 when the instrument 3 is to be removed.

In the exemplary embodiment shown, the spring clips 4-12 bound a receiving space 15 in which the instrument 3 is stored.

It can be seen here that the instrument 3 can be removed from the receiving space 15 only in one direction (upward in FIG. 1) because the spring clips 4-12 bound the receiving space 15 in the remaining five directions 16.

Each spring clip 4-11 is supported on both sides of its respective contact portion 13 at at least two points 17 and is fastened to the inner part 14.

These points 17 can be considered to be ends 18 of the contact portions 13.

The instrument 3 has a circumference that runs around an insertion direction 19.

The spring clips 4-12 are arranged in such a way that they surround the instrument 3, and therefore the spring clips 8 and 9 form an encircling spring clip.

In this case, the spring clips 7, 9, 11, on the one hand, and the spring clips 8, 10, on the other hand, are clamped over the inserted instrument 3.

The spring clips 7, 9 and 11, on the one hand, and the spring clips 8 and 10, on the other hand, are spaced apart from one another and in each case form a grid 20, 21.

The grids 20, 21 are assembled to form the inner part 14 and thus form a cage 22.

Here, for example, the encircling spring clip from the spring clips 8, 9 intersects the spring clip from the spring clips 6, 12.

The spring clips 7, 8, 9, 10, 11 also spread two longitudinally extending frame parts 34 of the inner part 14 apart, as a result of which the inserted inner part 14 is held in the housing 2.

The inner part 14 is produced here integrally as an injection-molded part, and therefore the spring clips 4-12 are nonreleasably connected to one another in an integrally bonded manner.

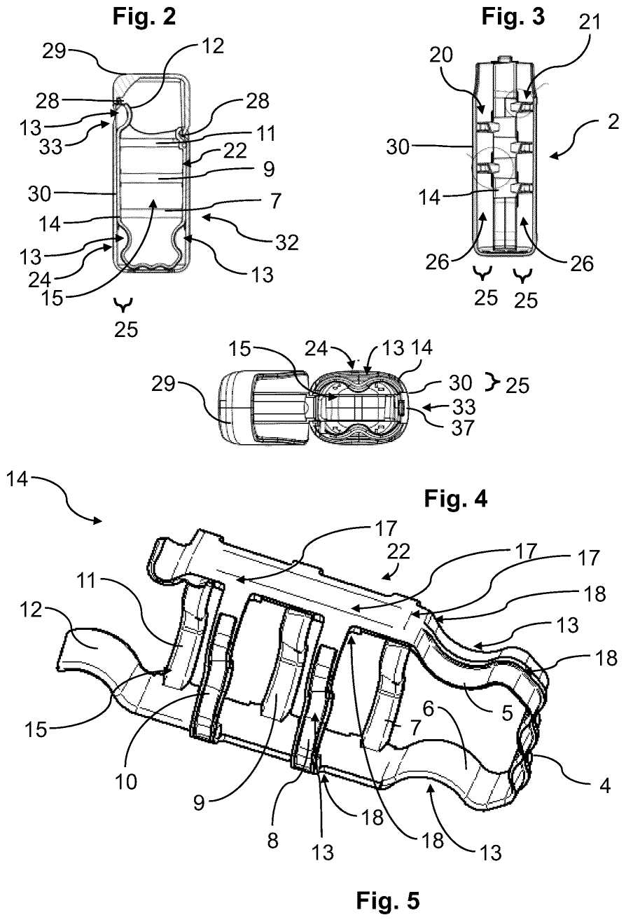

In the sectional illustrations according to FIG. 2 to FIG. 4, it can be seen that the spring clips 4-12 are each supported on the housing 2. By this means, a distance 25 is formed and spanned between a surface 23 of the instrument 3 inserted into the receiving space 15 and the housing 2, in particular a location 24 on the housing 2 that is opposite a contact portion 13.

This distance 25 defines a buffer zone 26, which, on the one hand, allows space for instruments 3 of differing sizes and, on the other hand, a means for shock absorption under mechanical stress of the housing 2 from the outside. The buffer zone 26 completely surrounds the inserted instrument 3.

The spring clips 4-12 and the inner part 14 as a whole have a plastics surface 27 which can bear against the instrument 3 without scratching the instrument 3.

In the exemplary embodiment, the entire inner part 14 is manufactured from the plastics material, but it can be provided in further exemplary embodiments that the spring clips 4-12 are manufactured from metal, for example, and are coated with plastic at least in the region of the contact portions 13 to form the plastics surface 27.

In the exemplary embodiment shown, the housing 2 is dimensionally stable in its entirety, while the inner part 14 allows a change in shape.

The housing 2 has a hinge 28, which connects two housing shells 29 and 30 to one another in an articulated manner.

The upper housing shell 29 serves here as a cover for the larger, lower housing shell 30. The lower housing shell 30 takes up more than three quarters of a surface of the housing 2.

As can be seen in FIG. 2, the inner part 14 is accommodated by the lower housing shell 30.

When being packed for the first time, the instrument 3 is first of all inserted into the receiving space 15 before the inner part 14 is moved into the housing 2. Thus, the inner part 14 is deformed before it is introduced into the housing 2.

During further use, the instrument 3 is removed from the inner part 14, the inner part 14 remaining in the housing 2, where it has been fixed in place.

The instrument 2 is thus accommodated with a predominant portion of its longitudinal extent 31 in the lower housing shell 30. In the process, the spring clips 4-12 are deformed further.

By clamping of the spring clips 4-12 between the instrument 3 and the housing 2, the inner part 14 maintains its position even when the housing 2 is open.

On the lower housing shell 30, a holding region 32 is formed with which the housing shell 30 can be held one-handed, for example between the palm of the hand and the index finger, middle finger, ring finger and/or little finger.

On the lower housing shell 30, facing away from the hinge 28, an actuating region 33 is formed, on which a finger of the hand holding the lower housing shell 30 can engage to pivot the upper housing shell 29 in the hinge 28 and open the housing 2 with one hand.

The actuating region 33 is used to actuate a latching lug 37, which holds the upper housing shell 29 in the unloaded state. When the latching lug 37 is released, the upper housing shell 29 pops up. One-handed opening is thus easy to carry out.

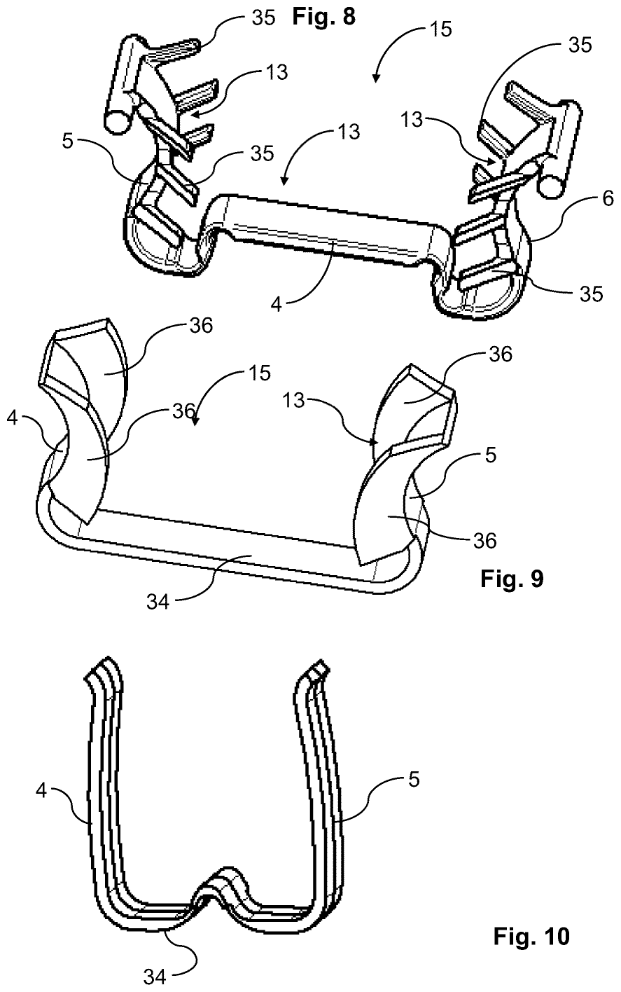

FIG. 8 shows another example of an arrangement of three spring clips 4, 5, 6, as can be used on an inner part 14 for a housing 2 according to FIGS. 1 to 7 or on another housing 2.

The spring clips 4, 5 have lateral contact projections 35 in their respective contact portions 13. Said contact projections 35 have free ends which can nestle against the instrument in question.

The receiving space 15 is bounded in three directions by the spring clips 4, 5, 6.

Otherwise, the explanations regarding FIGS. 1 to 7 apply correspondingly.

FIG. 9 shows another example of an arrangement of two spring clips 4, 5, as can be used on an inner part 14 for a housing 2 according to FIGS. 1 to 7 or on another housing 2, also in combination with the spring clips 4, 5 according to FIG. 8.

The spring clips 4, 5 have lateral contact flanges 36 in their respective contact portions 13. Said contact projections 36 have free ends which can nestle against the instrument in question.

Otherwise, the explanations regarding FIGS. 1 to 8 apply correspondingly.

FIG. 10 shows another example of an arrangement of spring clips 4, 5, as can be used on an inner part 14 for a housing 2 according to FIGS. 1 to 7 or on another housing 2, also in combination with the spring clips 4, 5 according to FIG. 8 and/or FIG. 9.

The spring clips 4, 5 are connected by a curved portion which contributes to the spring force between the spring clips 4, 5.

The arrangement according to FIG. 10 can be fixedly connected to a housing 2 or inserted into such a housing.

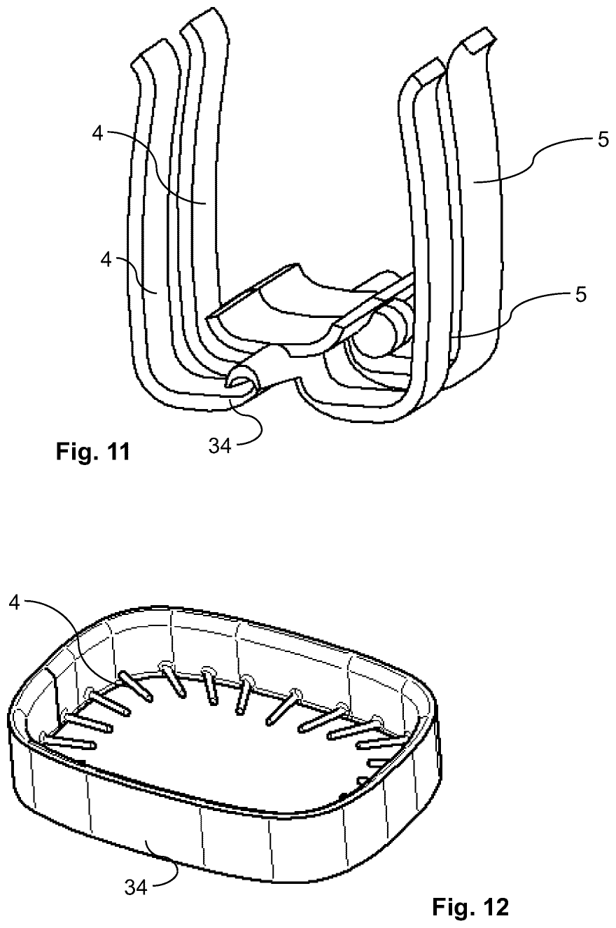

The spring clips 4, 5 according to FIG. 10 can be connected together in series in an arrangement according to FIG. 11 in order to fill a housing and to receive an instrument 3, in particular transversely with respect to its longitudinal extent 31.

FIG. 12 shows another example of an arrangement of spring clips 4, 5, as can be used on an inner part 14 for a housing 2 according to FIGS. 1 to 7 or on another housing 2, also in combination with the spring clips 4, 5 according to FIGS. 8 to 11.

The spring clips 4, 5 are formed with an annular frame part 34, similarly to the contact projections 35 according to FIG. 8.

The annular frame part 34 can be arranged, for example, in a housing 2.

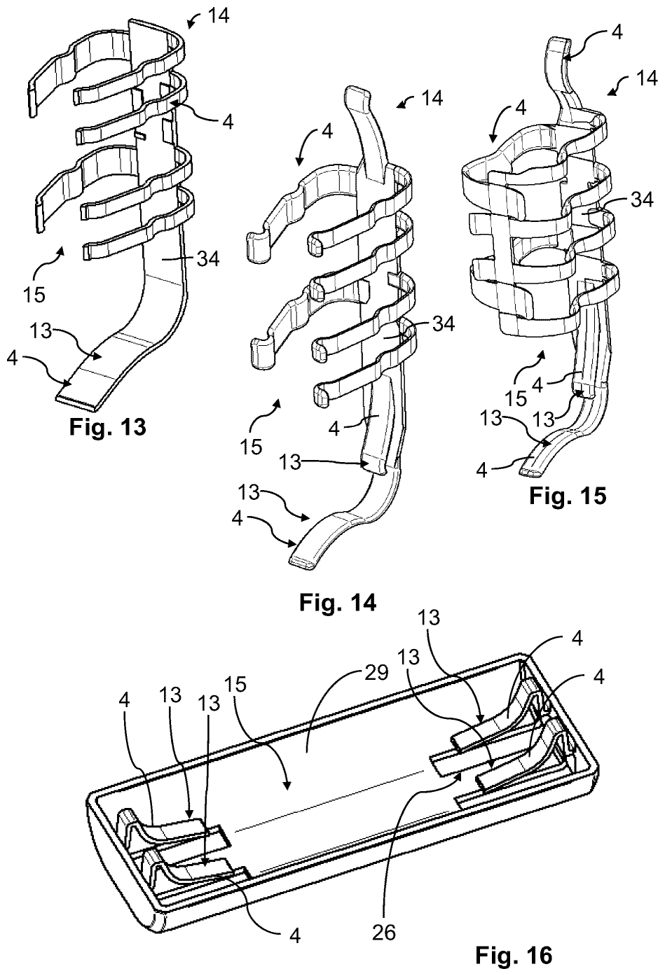

FIGS. 13 to 15 show further examples of an arrangement of spring clips 4, 5, as can be used on an inner part 14 for a housing 2 according to FIGS. 1 to 7 or on another housing 2, also in combination with the spring clips 4, 5 according to FIGS. 8 to 12.

The inner parts 14 according to FIGS. 13 and 14 bound the receiving space 15 in four directions, with the variant according to FIG. 14 allowing insertion of the instrument from two directions.

FIG. 16 shows another example of an arrangement of spring clips 4, as can be used in a housing 2 according to FIGS. 1 to 7 or in another housing 2, also in combination with the spring clips 4, 5 according to FIGS. 8 to 15.

The spring clips 4 are formed integrally on a housing shell 29 of the housing 2. Another housing shell 30 is connected in an articulated manner to the housing shell 29 to complete the housing 2.

In the exemplary embodiments shown, the housing shells 29, 30 are connected by a hinge 28 to form a flip-down case. In further exemplary embodiments, the housing shells are formed displaceably relative to one another, for example to form a sliding case.

In the case of an instrument holder 1, it is proposed, for the flexible holding of different instruments 3 within a housing 2, to form at least one spring clip 4-12 which contacts and/or acts upon the instrument 3 inserted in each case.

### LIST OF REFERENCE SIGNS

1 instrument container
2 housing
3 instrument
4-12 spring clips
13 contact portion
14 inner part
15 receiving space
16 direction
17 point
18 end
19 insertion direction
20 grid
21 grid
22 cage
23 surface (of the instrument)
24 location
25 distance
26 buffer zone
27 plastics surface
28 hinge
29 (upper) housing shell
30 (lower) housing shell
31 longitudinal extent of the instrument
32 holding region
33 actuating region
34 frame part
35 contact projection
36 contact flange

1. An instrument container, comprising:
a housing; and
at least one spring clip located at least one of on or in the housing which is designed to bear against an inserted instrument.

2. The instrument container as claimed in claim 1, wherein the at least one spring clip is strip-shaped at least in a contact portion that is adapted to contact the inserted instrument.

3. The instrument container as claimed in claim 1, wherein the at least one spring clip is formed on an inner part that is locatable in the housing.

4. The instrument container as claimed in claim 3, wherein the at least one spring clip is arranged removably on or in the housing as part of the inner part which is removable from the housing.

5. The instrument container as claimed in claim 1, wherein the at least one spring clip bounds a receiving space for the instrument in at least two directions.

6. The instrument container as claimed in claim 3, wherein the at least one spring clip is at least one of fastened or supported at least at one of ends thereof or at least two points thereon on the removable inner part.

7. The instrument container as claimed in claim 1, wherein the at least one spring clip includes at least two of the spring clips arranged in an encircling manner.

8. The instrument container as claimed in claim 1, wherein the at least one spring clip is formed on includes a plurality of the spring clips arranged as a grid and form a cage.

9. The instrument container as claimed in claim 1, wherein the at least one spring clip comprises a first spring clip and at least one further spring clip which are designed to bear against an inserted instrument, and at least one of a) the further spring clip is connected to the first spring clip, or the first spring clip and the further spring clip are arranged in the housing such that the first spring clip and the further spring clip clamp each other over an inserted instrument.

10. The instrument container as claimed in claim 1, wherein the at least one spring clip is supported on the housing.

11. The instrument container as claimed in claim 1, wherein the at least one spring clip spans a distance between the housing and an inserted instrument.

12. The instrument container as claimed in claim 1, wherein the at least one spring clip has a surface formed of plastic.

13. The instrument container as claimed in claim 3, wherein the inner part having the at least one spring clip is at least one of formed integrally or as an injection-molded part.

14. The instrument container as claimed in claim 1, wherein the housing is dimensionally stable.

15. The instrument container as claimed in claim 1, wherein the housing has at least two housing shells which are connected to each other.

16. The instrument container as claimed in claim 15, wherein at least one of the housing shells of the housing forms more than one half of a surface of the housing.

17. The instrument container as claimed in claim 15, wherein the at least one spring clip is substantially or completely accommodated by one of the at least two housing shells.

18. The instrument container as claimed in claim 1, wherein the at least one spring clip creates a receiving space which holds an inserted instrument at least one of a) on all sides at a distance from the housing, b) at least over a longitudinal extent of the instrument.

19. The instrument container as claimed in claim 1, wherein the housing has at least first and second housing shells which are movable relative to one another, and at least one of the first housing shell has a holding region for contacting by a hand or the second housing shell has an actuating region for contacting by a finger of a hand.

20. The instrument container of claim 1, wherein the at least one spring clip is configured to accommodate instruments of differing shapes and/or differing sizes.

21. A method for at least one of packing or transporting an instrument in a housing, the method comprising:
introducing the instrument into a receiving space of an inner part which has at least one spring clip by deforming the at least the at least one spring clip; and
introducing the instrument with the inner part into the housing, with the at least one spring clip being further deformed; or
introducing the inner part into the housing with the at least one spring clip being deformed by the inner housing; and
introducing the instrument into the inner part in the housing with instrument deforming the at least one spring clip.
